(19) United States
(12) Patent Application Publication
(10) Pub. No.: US 2017/0281536 A1
(43) Pub. Date: Oct. 5, 2017

(54) DEGRADABLE NETWORKS FOR SUSTAINED RELEASE AND CONTROLLED RELEASE DEPOT DRUG DELIVERY APPLICATIONS

(71) Applicant: Lance L. GOOBERMAN, Merchantville, NJ (US)

(72) Inventor: Lance L. GOOBERMAN, Merchantville, NJ (US)

(21) Appl. No.: 15/086,756

(22) Filed: Mar. 31, 2016

(51) Int. Cl. A61K 9/00; A61K 31/145; A61K 47/34; A61K 31/485

(52) U.S. Cl. CPC A61K 9/0024; A61K 31/485; A61K 31/145; A61K 47/34

(57) ABSTRACT

Provided herein is a controlled release and/or sustained release depot drug delivery system, comprising, a biodegradable polymer coating and an API coated with the biodegradable polymer coating, wherein a quantity of API coated with biodegradable polymer is effective to be released. from the biodegradable polymer coating over a prolonged period of time.

### FIELD OF THE INVENTION

The present invention broadly concerns the production and application of degradable polymeric network compositions for controlled and sustained release of drug delivery systems, and in preferred embodiments concern poly (lactic acid) and poly (glycolic acid) and related co-polymeric composite networks for controlled and sustained release delivery of opiate and/or opioid antagonists and other addiction combating and behavioral disorder active pharmaceutical ingredients, and particularly to novel related drug depot implant designs and applications.

### BACKGROUND OF THE INVENTION

There are several methods of injection or infusion of active pharmaceutical ingredients (“API”) used in humans, including intradermal, subcutaneous, intramuscular, intravenous, intraosseous, intraperitoneal, intrathecal, epidural, intracardiac, intraarticular, intracavernous, and intravitreal. Time release or otherwise longer acting forms of subcutaneous/intramuscular injections are available for various drugs, and are typically referred to as depot injections.

Various drugs or therapeutic agent depot systems and applications are known and have been used with varying success in a variety of diverse applications. For example, U.S. Pat. No. 6,203,813 provides for a pharmaceutical delivery device and method of preparation therefor, the entire disclosure of which is herein incorporated by reference. Among other embodiments, this device provides for an opiate antagonist implant which is an admixture of an opiate antagonist and a pharmaceutically acceptable carrier compressed into a subcutaneously implantable pellet. The pellet is effective to release levels of opiate antagonist over the desired amounts of time when implanted in a patient which can effectively inhibit the effects of a number of additive drugs.

U.S. Pat. No. 7,736,665 discloses an implantable biocompatible nonerodible ethylene vinyl acetate co-polymeric matrix (PVA) for releasing buprenorphine through pores that open to the surface of the polymeric matrix in which it is encapsulated for the sustained release of the drug for treatment of opiate addiction and pain.

U.S. Pat. No. 7,741,273 also discloses drug depot implant designs for sustained release of therapeutic agents to alleviate pain in subjects associated with neuro-muscular and skeletal injury or inflammation. These delivery designs rely on a body portion in the form of an implantable polymetric matrix for the extended release of therapeutics into spinal areas, shoulders, hips, or other joints. Implant materials described for use in this system include, for example, hydrophilic materials, such as hydrogels, or may be formed from biocompatible elastomeric materials, such as, including silicone, polyisoprene, copolymers, of silicone and polyurethane, neoprene, nitrile, vulcanized rubber and combinations thereof, Some hydrogels are described as natural hydrogels, and those formed from polyvinyl alcohol, acrylamides such as polyacrylic acid and poly (acrylonitrile-acrylic acid), polyurethanes, polyethylene glycol, poly(2-hydroxy ethyl methacrylate) and copolymers of acrylates with N-vinyl pyrolidone, N-vinyl lactams, acrylamide, polyurethanes, and polyacrylonitrile or may be formed from other similar materials that form a hydrogel.

As described, the hydrogel materials may further be cross-linked to provide further strength to the implant. Examples of different types of polyurethanes include thermoplastic or thermoset polyurethanes, aliphatic or aromatic polyurethanes, polytherurethane, and polycarbonate-urethane and silicone polyether-urethane. Other mentioned hydrophilic polymers said to be useful include naturally-occurring materials such as glucomannan gel, hyaluronic acid, polysaccharides, such as cross-linked carboxyl-containing polysaccharides, and combinations thereof.

Also described are therapeutic agents such as an anti-inflammatory agent or osteoinductive factor, and packaged in gas-filed lipid-containing microspheres which may further comprise biocompatible polymers on their outer surfaces. These polymers are said to include, for example, natural occurring polysaccharides, such as for example, arabinans, fructans, fucans, galactans, galacturonans, glucans, mannans, xylans, such as, inulin, levan fucoidan, carrageenan, galatocarolose, pectic acid, pectin, amylase, puliulan, chitin, agarose, keratin, chondroitan, dermatan, hyaluronic acid, alginic acid, xanthan gum, starch and various other natural homopolymer or heteropolymers such as those containing one or more of the following aldoses, ketoses, acids or amines; erythrose, threose, ribose, arabinose, xylose, lyxose, allose, glucose, mannose, gulose, idose, galactose, talose, erythrulose, ribulose, xylulose, psicose, fructose, sorbose, tagatose, manitol, sorbitol, lactose, sucrose, trehalose, maltose, cellobiose, glycine, serine, threonine, cysteine, tyrosine, asparagine, glutamine, a.spartic acid, glutamic acid, lysine, arginine, histidine, glucuronic acid, gluconic acid, glucaric acid, g alacturonic acid, mannuronic acid, glucosamine, galactosamine, and neuraminic acid, and naturally occurring derivatives thereof. Exemplary semi-synthetic polymers include carboxymethylcellulose, hydroxymethylcellulose h droxyproplmethylcellulose, methylcellulose, and methoxyceliulose. Exemplary synthetic polymers include polyethylenes, such as, polyethylene glycol, polyoxyethylene., and polyethylene terephthalate; polypropylenes, such as, polypropylene glycol; polyurethanes, such as, polyvinyl alcohol (PVA), polyvinylchloride and pot inylpyrrolidone; polyamides including nylon, polystyrene, polylactic acids; fluorinated hydrocarbons, and fluorinated carbons, such as, Polytetrafluoroethylene; and polymethylmethacrylate, and derivatives thereof. Also described are extended-release and. sustained-release compositions which can be administered in a controlled and sustained manner by implanting a therapeutic agent dispersed within a polymer matrix depot that breaks down over time within tissues, or which is incorporated within a protective coating to provide delayed release of the therapeutic agent. Examples of polymer matrixes include the biopolymers poly (alpha-hy- droxy acids), poly (lactide-co-glycolide) (PG), polyethylene glycol (PEG) conjugates of poly (alpha- hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-P AA copolymers, ALGA-PEO-PLGA, polyphosphoesters, polya.nhydrides, polyester-anhydrides, polya.mino acids, polyurethane-esters, polyphospha.zines, polycaprolactones, polytrimethylene carbonates, pobidixanones, polyamide-esters, polyketals, polyacetals, glycosaminoglycans, hyaluronic acid, hyaluronic acid ester carbonates, polydesamnotyrosine ester arylates, polyurethanes, polypropylene fumarates, polydesaminoty-rosine ester carbonates, polydesanmotyrosine ester arylates, polyethylene oxides, polyorthocarbonates, polycarbonates, or copolymers or physical blends thereof or combinations thereof. The biopolymer may also provide for non-immediate (i.e., sustained) release. Examples of suitable sustained-release biopolymers include, poly(alpha-hydroxy acids), poly(lactide-co-glycolide) (PLGA), polyactide (PLA), polygycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polya.cticve,), methacrylates, poly(N-isopropylacrylainide), PEO-PPO-PEO (pluronics), PEO-PPO-PAA copoly ners, PLEA-PEO-PLEA, or combinations thereof. See also U.S. Pat. No. 7,727,954.

U.S. Pat. No. 6,835,194 also discloses an implantable drug delivery system to deliver fentanyl or a fentanyl congener over a protracted period of time. Here, rather then employing a polymeric metix depot, there is described a housing containing a reservoir, which contains a drug formulation and a pump connected to the housing to facilitate movement of the drug from the reservoir. There is also a thermal expansion element to provide for a flow pathway by way of a thermal expansion channel.

While most, if not all, of such described drug delivery methods and systems, and others not mentioned herein, are no doubt effective and desirable to at least some degree, under appropriate circumstances, there remains many areas for improvement in depot drug or therapeutic product delivery, inclusive of both pharmaceutical delivery methods and systems and materials employed.

### SUMMARY OF THE INVENTION

In accordance with the present invention, there is provided a novel and superior sustained release and/or controlled release drug delivery system, product, methods of manufacture and methods of use comprising in its broadcase sense a controlled release and/or sustained release system comprising a biodegradable polymer coating material and a quantity of a pharmaceutical active ingredient or API coated with the polymeric material, and in which fractioned amounts of API can he released upon degradation of the polymeric network coating, by way of a timed combination of polymer coating degradation and API dissolution to deliver controlled effective dosages of API over a desired time period, such as over weeks and/or months, or even over a year or more to achieve a desired therapeutic effect. The invention is also particularly effective for controlled and sustained release of addiction combatting and behavior disorder API and to novel related drug implant designs and applications.

The invention will be more fully explained with reference to the following detailed description with accompanying claims.

### BRIEF DESCRIPTION OF THE DRAWINGS

FIG. 1 illustrates example images of loose film polymer longevity in accordance with an embodiment of the invention.

FIG. 2 illustrates example images of loose film polymer degradation in accordance with an embodiment of invention.

FIG. 3 is an illustrative study of triamcinolone release from wrapped and loose pellets in accordance with an embodiment of the invention.

FIG. 4 illustrates example images of loose film polymer degradation in accordance with an embodiment of the invention.

FIG. 5 illustrates example images of loose film polymer degradation in accordance with an embodiment of the invention.

FIG. 6 is an illustrative study of triamcinolone release form wrapped and loose pellets in accordance with an embodiment of the invention.

### DETAILED DESCRIPTION

The present invention provides in its broadest sense the production and application of degradable depot implantable network systems for the controlled and sustained release of drug delivery systems, inclusive of a wide variety of drugs and/or otherwise therapeutically active compounds and substances effective to treat or act up upon a wide variety of externally and/or inter rally effected or affected afflictions, maladies, disorders and the like, such as described in detail in U.S. Pat. No. 6,203,813, the entirety of which is incorporated herein by reference, and which therapeutically effective substance(s) will generally be referred to herein as “drug delivery” or “drug delivery systems” or “active pharmaceutical ingredient” (“API”). Also incorporated herein by reference in their entirety are U.S. Pat. Nos. 6,203,813, 6,004,962 and 5,789,411 . The degradable depot implantable drug delivery systems of the intention have been found to be particularly effective in their controlled and sustained release properties, such a.s, for example, in emitting or delivering contemplated dosages of API over contemplated extended time periods in a. consistent, predictable and non-hazardous fashion by employing co-polymeric networks of poly (lactic acid) (“PLA”) and/or poly (glycolic acid) (“PGA”) and copolymers thereof encapsulating and/or otherwise coating a. desired APL The degradable network of the polymeric materials employed to coat or encapsulate an API for controlled degradation of polymeric network and for controlled and sustained release delivery of an API can be controlled in accordance with the invention to any desired degree to allow for the desired precision timed and dosage release of a desired or contemplated API, and to achieve the contemplated beneficial results as described above, such as, for example, thirty, sixty and ninety-da.y and longer time release periods, such as six months to over a year, by the particular composition of polymeric coating employed to coat a particular API or API composition.

Such PLA/PGA biodegradable polymers and their associated copolymeric networks have been known for their role in medical applications, especially i n orthopedic applications, as they degrade by hydrolysis and enzymic activity and have a range of mechanical and physical properties that can be engineered appropriately to suit a particular application, for example, as sutures and osteosynthesis devices. See, for example, Ashammakhi, N. et al., “Absorbable polyglycolide devices in trauma and bone surgery”, Biomaterials, 1997, 18 (1): pp. 3-9. See also, for example, Xu H. et al., Rapid prototyped. PGAIPLA scaffolds in the reconstruction of mandibular condyle bone defects, Int. J. Robot, 2010 March; 6 (1): 66-72. See further, for example, with respect to some difkrent biodegradable hydro gels as used in drug administration mechanisms, poly (ethylene glycol) (PEG) degradable networks. Drexel University Ph.D thesis of J. L. Ostroha, June 2006, disclosing PEG aqueors resistant degradation compounds complexed with hyduolysis degradable PLA as co- polymers. However, a.s controlled release drug delivery systems health issues and biocompatibility issues of PEG/PLA complexes remain uncertain.

With respect to known biocompatible and safe PGA and PLA complexes, without desiring to limit this invention to any particular theory, degradation characteristics are generally thought to depend on several parameters, including, without limitation, molecular size, molecular structure, crystallinity, and copolymer ratios. These biomaterials are also rapidly gaining recognition in the fledging field of tissue engineering because they can be fashioned into porous scaffolds for carriers of cells, extracellular matrix components, and bioactivc. agents.

In the context of this invention, there is a comprehensive review of properties and applications of biodegradable PLAIPGA polymers and their copolymers as used to formulate implantable depot controlled and sustained release delivery systems of API, such as disclosed in U.S. Pat. No. 6,203,813, and especially in a preferred embodiment opioid antagonists and other addiction-combattima effective APis. As used herein the terms PLA, PGA, or PLA-PGA do not denote one material, but rather a large family of materials inclusive of difkrent stereochemical structures that have a wide range of differing bioengineering propel lies and concomitant biological responses.

As mentioned, PLA and PGA and their related complexes have been studied for several biomedical applications because of their known biocompatibility, bioresorbabil ity and safety of use. See, erg. Kim, K., et al., “Control of degradation rate and hydrophilicity in electrospun non-woven poly (lactide) nano fiber scaffolds for bin-medical applications”, Biomaterials 2003, 24 (27): pp. 4977-4985 and Seppala, Jukka et al., “Degradable Polyesters through chain linking for packaging and Biomedical Applications”, Macromolecular I3ioScience 2004, 4 (3): pp. 207-21.7. However. as PLA is known as a somewhat hydrophobic and brittle polymer, albeit degradable via hydrolysis of the lactic acid group, it has been primarily known for use in tissue engineering applications where rigid scaffolds are formed for the cultivation of cells, rather than as a depot implantable drug delivery mechanism or vehicle. See, e.g., Quirk, R. A. et aL, “poly (lysine) GRGDS as a biomimetic surface modifier for poly (lactic acid)” Biomaterials, 2001, 22 (8): pp. 865-872, Thus, PLA and PGA complexes ha been said to exhibit shortcomings as a material for many drug delivery applications, in at least one aspect which is attributed to their hydrophobic and rigid nature.

Despite the perceived shortcomings of the past, however, the present inventive products and methods provide novel uses for PLA and PGA copolymers and their polymeric conjugates and other copolymeric combinations as effective time release coatings for drug or API implantable depot delivery systems, especially for opioid and other addiction combating APIs, by exploiting the controlled degradation rates of particular copolymeric compositions , such as particular polymer ratios employed, and the degradation rates of particular API and/or API compositions employed therewith, which products, systems and methods and composition of manufacture and methods of use have heretofore been unknown.

By way of example, but not intending to be limiting in any way, a discussion of the biodegradable polymers and their conjugate networks employed in the present invention and other polymeric networks useful herein is in order. As mentioned, many biodegradable polymers have seen use in medical applications as surgical sutures, drug delivery systems, internal fixation devices and tissue engineering scaffolds, with some preferred in certain applications, such as PLA and PGA in tissue rebuilding moves, but essentially dismissed as drug delivery vehicles for, perhaps, path ally non-understood or less than well understood reasons. With respect to the two main types of biodegradable polymers, natural and synthetic, the synthetic polymers PLA and PGA and their copolymer poly (lactic- co-glycolic acid) (PLGA) may be employed herein in some preferred embodiments as implantabie depot drug delivery systems. Example methods of production and a. description of the structures of PLA, PGA and copolymeric conjugates follow, but again, such are merely supplied as examples and are not intended to limit the invention in any way, as useful, suitable polymeric materials described herein can be obtained from any source.

PLA is a biodegradable thermoplastic aliphatic polyester which is derived from renewable resources such as corn starch, or cane sugar. Usually, bacterial fermentation is used to produce lactic acid. As an example, in the preparation of PLA, lactic acid is first oligomerized, and then catalytically dimerized into the lactide monomer, which is a diester ring, which is followed by ring-opening polymerization.

PGA is a biodegradable thermoplastic linear, aliphatic polyester, and in preparation glycolic acid can be reacted into a glycolide, which can then be polymerized using a ring-opening polymerization. Another simple process is to use a condensation reaction to polymerize glycolic acid; however, this process yields a lower molecular weight polymer than the ring-opening polymerization due to the side products of the condensation reaction. The ring opening polymerization is a multistep process that begins with the low molecular weight lactic or glycolic acid polymer. After the low molecular weight polymer is obtained, the glycolide is distilled by heating at low pressure. Although there are a variety of catalysts available such as antimony compounds, zinc compounds, and alkoxides, stannous octoate is preferred because it is approved by the FDA as a food stabilizer. The ring-polymerization is typically started with the initiator and glycolicle or lactide at a temperature of approximately 195° C. After some time, such as two hours or so at this temperature, the temperature is increased, typically to approximately 230° C. After the polymer solidifies, a higher molecular weight polymer may be obtained. Another known way to prepare the polymer is to induce a solid-state polycondesation of halogenoacetates. After heating the halogenoacetate typically between approximately 160 and 180° or so under nitrogen, poluglycolide is formed. The side-product salts are removed by washing the polymer with water.

The copolymer, poly (lactic-co-glycolic acid) (PLGA), al s o useful in the present invention, can be produced using the same techniques.

Polymerization of a racemic mixture of L- and D-lactides usually leads to the synthesis of poly-DL-lactide (PDLA). Use of stereospecific catalysts can lead to heterotactic PLA which has been found to show crystallinity, as opposed amorphous polylactide.. The degree of crystallinit, and many important properties, is thought to be largely controlled by the ratio of D to L enantiomers used, and to a lesser extent on the type of catalyst used. Due to the chiral nature of lactic acid, several distinct forms of polylactide exist, with, for example, poly-L-lactide (PLLA) being the product resulting from polymerization of L, L-lactide (also known as L-lactide), and which PLLA has a crystallinity of around 37%, a glass transition temperature between 60-65° C., a melting temperature between 173-1 78° C. and a tensile modulus between 2.7-16 GPa.

Polylactic acid can be processed like most thermoplastics into fiber and film. The melting temperature of PLLA can be increased 40-50° C. and its heat deflection temperature can be increased from approximately 60° C. to up to 190° C. by physically blending the polymer with PDLA (poly-D-lactide). PDLA and PLLA are known to form a highly regular stereocomplex with increased crystallinity. The temperature stability can be maximized when a 50:50 blend is used, but even at lower concentrations of 3-10% of PDLA, there is still a substantial improvement. In the latter use, PDLA is thought to act as a nucleating agent, thereby increasing the crystallization rate. Biodegradation of PDLA is slower than for PLA due to the higher crystallinity of PDLA.

Polyglycolide is thought to have superior properties to that of other synthetic drug delivery/biocompatible polymers, partly due to its greater stereoregularity. Polyglycolicle has a glass transition temperature between 35-40° C. and its melting point is reported to be in the range of 225- 2300C. PGA also exhibits an elevated degree of crystallinity, around 45-55%, thus resulting in insolubility in water. The solubility of this polyester is somewhat unique, in that its high molecular weight form is insoluble in almost all common organize solvents (acetone, dichloromethane, chloroform, ethyl acetate, tetrahydrofuran), while low molecular weight oligomers sufficiently differ in their physical properties to be more soluble. However, polyglycolide is soluble in highly fluorinated solvents like hexafluoroisopropanol (HEW) and hexafluoroacetone sesquihydrate, which can be used to prepare solutions of the high MW polymer for melt spinning and film preparation. Fibers of PGA exhivit high strength and modulus (7 GPa) and are particularly stiff.

While all PLGA copolymers have a glass transition temperature between 40 and 60° C., their melting point and percent crystallinity depend on the percent composition. For example, a 5:95 ratio of PGA to PLA copolymer has a melting temperature of 173° C. while a 90:10 ratio of PGA o PLA copolymer has a. melting point of 201° C.

Polylactide can be processed in similar manner as many other thermoplastics, for example, into fiber using a traditional melt spinning process; however as one increases the percent composition of PDLA, its crystallinity increases, making it slightly more difficult to process.

Polyglycolide is thought to present some problems with processing due to its high melting temperature of 220° C. To overcome this. blends and copolymers are typically used, with a. common copolymer of PGA being a 50/50 copolymer with lactic acid. referred to as PLGA. Currently PLGA is mostly produced for use in drug delivery systems. To do so, PLGA and the drug are dissolved in a. solvent, such as dichloromethane (DCM). Upon solvent evaporation, microparticles of the PLGA form, and such are usually further cast to obtain drug-loaded, or otherwise drug incorporated polymer films to be used in drug delivery systems or drug incorporated polymeric microspheres.

Owing to its hydrolytic instability, however, the use of polyglycolide has initially be limited. Currently polyglycolide and its copolymers (polyla.ctic-co-glycolic acid) with lactic acid, poly (glycolide-co-caprolactone) with c-caprolactone. and poly (glycolide-co-trimethylene carbonate) with trimethylene carbonate) are widely used as a material for the synthesis of absorbable sutures, and have being evaluated in the biomedical field.

Other contemplated biodegradable polymers possibly useful in the present invention include, for example, without limitation and in addition to, poly(lactides), and poly(glycolides), poly(lactide-co-glycolides), poly(lactic acid)s, poly-(glycolic acid)s, poly(lactic acid-co-glycolic acid)s, polycaprolactone, polycarbonate,s, polyesteramides, polyanhydrides, poly(amino acids), polyorthoesters, polycyanoarcylates, poly(p-dioxanone), poly(alkylene oxalates), biodegradable polyurethanes, blends and copolymers thereof.

Polylactide is known to take a long time to degrade biologically, with a biodegradation time possible of more than two years , Within different types of PLA, the stereo form of PIA, PDLA degradation is known to be more slowly due to its higher crystallinity. In contrast, polyglycolide is known to biodegrade relatively quickly, such as sometimes in only two to three months. Thus, through research and development, and extensive experimentation with particular blends, such as in accordance with the present invention, particular polymeric mixtures have proven to be surprisingly beneficial and/or efficacious in providing time-release of coated API as desired. and which will be refened to herein as engineered polymeric blends. :Polyglycolide is known to be degraded by random hydroysis into glycolic acid. According to some studies, there is thought to be a two-step process: first, water diffuses into the amorphous regions and cleaves the ester bonds there. After these regions are eroded, the crystalline regions are also dissolved. See Middleton, J. et al., “Synthetic Biodegradable Polymers as Medical Devices,” Medical Plastics and Biomaterials Magazine, 1998. Relative to PLA, PGA dissolves much quicker. In studies with medical sutures, PGA has been shown to lose 50% of its strength in only two weeks, and all after a month.

Because these polymers vary differently in the biodegradation time, copolymers can be produced with a wide-variety of degradation times, depending on the contemplated use, by varying the percent composition of the copolymers.

Thus, the present invention provides a novel delivery system comprising a pharmaceutical/biologically active substance, or API, and a pharmaceutically acceptable coating on the API to form an implantable depot pellet for deployment in a patient, and effective for the delivery of therapeutically effective levels of the desired API over desired extended periods of time. The API coated depot implants of the invention herein here been found to be superior in time release and controlled release of API compared to known materials of incorporating an API throughout a polymeric matrix, such as in microspheres, rather than coating an API with a specifically engineered polymeric time- release degradable coating such as in the present invention.

By “API” or “phannaceuticallbiologically active substance” or otherwise “active pharmaceutical ingredient” as used herein is meant any conventional, experimental, novel or as yet unknown pharmaceutical, drug or biologically active substance for use in animals or humans, such as, for example, and without limitation, described in U.S. Pat. No. 6, 203,813. Drugs can be in various forms, such as uncharged molecules, components of molecular complexes, or nonirritating, pharmacologically acceptable salts such as hydrochloride, hydrobromide, sulfate, phosphate, nitrate, borate, acetate, maleate, tartrate, salicylate, etc. For acidic drugs, salts of metals, amines, or organic cations (e.g., quaternary ammonium) can be employed. Furthermore, simple derivatives of the drugs (such as ethers, esters, amides, etc.) which have desirable retention and release characteristics but which are easily hydrolyzed by body pH, enzymes, etc., can be employed.

The amount of drug or bioactive substance incorporated in the drug-delivery device of the invention can vary widely depending on the particular drug, the desired therapeutic depending on the particular drug, the desired therapeutic effect, and the time span for which it takes the subcutaneously implantable pellet to erode or dissolve. Since a variety of the inventive devices in a variety of sizes and shapes are intended to provide complete dosage regimes for therapy for a variety of maladies, there is no critical upper limit on the amount of drug incorporated in the device. The lower limit also will depend on the activity of the drug and the time span of its release from the device. Thus, it is not practical nor necessary to define a range for the therapeutically effective amount of drug to be release by the device.

Other examples of active ingredients useful in the present inventive devices include medicaments which are effective in a small amount or in any amount, and in which their activity is promoted by sustained release, and additionally medicaments which are unstable to heat. Some other non-limiting examples of such active APIs useful in this invention, which are limited really by one's own imagination, are disuifiram, clonidine, ⁻buprenorphine, varenicline, nicotine, psychoactive substances like selective serotonin re- uptake inhibitors, antidepressants, mood stabilizers, stimulants, sedatives, anxiolytics, anti-psychotics, as well as antibiotics, anti-infectives, anti-fungal agents, and anti-viral drugs, antihypertensives, compounds to lower cholesterol levels such as statins, birth control medications, compounds to control gene disorders such as sickle cell anemia or to treat the effects thereof, various sedatives and pain medications,anti-epileptics, hormones, bronchodilators, vasodilators, vasoconstrictors, decongestants, hypoglycemic agents, bariatric medications, gastric acid reducing agents, antidiarrheal agents, anti-inflammatory agents, tissue plasminogen activator, prostaglandins, prostacyclines, various bio- hormones, interferons, interleukins, tumor necrosis factor, and some other cytokines (e,g. macrophage activating factor, migration inhibitory factor and colony stimulating factor). The bio-hormones means substances which are produced within the living body and regulate the bio-functions, and include growth hormone (GH) such as human growth hormone (HUM bovine growth hormone (bGH) including bio-synthetic product (B-HGH, etc.); growth hormone releasing factors (GRF) which are known as peptides consisting of a number of amino acids 44, 40, 37, or 29 (e.g. liGRF (1-44) NH2, liC3RF (1-29) NH-12); somatomedines (SM) such as SM-A, SM-B, SM-C, insulin-like growth factor (IGF)-1, IGF-II, and multiplication stimulating activity (MSA); and calcitonin (i.e. calcium regulating hormone secreted from the mammalian thyroid gland and in non-mammalian species from the ultimobranchial gland). The above active ingredients ma be used alone or in combination of two or more thereof.

Closely related are the addictive drug antagonists. (fan addictive drug such as heroin, morphine, codeine, neopine, etc. is taken while the blood still contains the antagonist, the addictive drug will pass through the body and be harmless to the taker in the sense that the taker will not experience “a high” and the drug will not be addictive. Such antagonists have offered a very successful method for treating drug addicts while the addicts are at clinics; however, it has been noted that once an addict returns to his original environment, and is out of control of the clinic, he is likely to stop taking the antagonist and resume taking one of the addictive drugs. For this reason, the implantable depot device of this invention offer unique advantages in treating drug addicts by this method, since the device containing an antagonist implanted with the addict's body provides him no control over the administering of the antagonist. thus extending the addict's period of cure beyond the time that he can actually be confined to a clinic. Some examples of specific drug antagonists suitable for incorporation into this inventive implant include N-allyinoroxymorphone (“naloxone”) and 2-cyclopropylmethyl-2′ hydroxyl-5, 9-dimethyl-6, 7-benzomorphone (“cyclazocine” and “naltrexone”).

Also contemplated for use herein are prodrugs and their biologically active metabolites either of which may he an effective therapeutic active ingredient in accordance with this invention, such as, for example, naltrexone and one of its pharmaceutically active metabolites six-naltrexone and disuifirom.

The above listines of drugs is not intended to be comprehensive but merely representative of the wide variety of drugs and biologically active substances, or APIs, which can be used with this invention. Those skilled in the art will know or be able to determine by routine experimentation that many other specific drugs are and/or biologically active substances also suitable.

As discussed hereinabove, the amount of drug dispersed in the inventive implant will depend, of course, on many factors including the specific drug, the function to be accomplished, the length of time it is desired to dispense the drug, the amount of drug to be dispensed in a specific time, the size of the device, and many other factors. In general, API amounts ranging from about 0.01% to about 99.9% by weight of the device can be coated with polymeric sustained release material to form the implantable depot drug delivery devices/products of the invention.

The amount of drug to be dispensed in a specific time, will of course, depend on such factors as the particular application, the particular drug, the age of the patient, etc. In general, vhat will constitute an “effective amount” will be known or easily ascertainable by those skilled in the art. Much of this type of data is published in the literature or easily determined by routine experimentation. Examples of the published literature on effective amounts of progestin-type steroids, in this case for topical application, can he found in Shipley, “Effectiveness of Topically Applied Progestational Agents,” Steroids 7 (4): 341-349, (April 1966). In a like manner, the following literature describes effective amounts of addictive drug antagonists: MARTIN, W. R., “Opioid Antagonists,” Parmacological Review s, Vol. 19 no. 4, pp. 463-521 (1967) and references contained therein; FREEDMAN, A. M., “Cyclazocine and Methadone in Narcotic Addiction,” The Journal of the American Medical Association. Vol. 202, pp. 191-194 (Oct. 16. 1967). Also, the patents mentioned above often contain data on effective amounts for any particular application.

In addition to the control over delivery of drugs which can be obtained through proper choice and design of the inventive implant as discussed supra, the dosage administered by this implant can be controlled by the size and shape of the implant device, concentration of the drug in the device, density of the device, and nature of the carrier surface area, pore size, matching of the carrier and drug, nature of the surroundings, etc. This is of a particular advantage where it is desirable to deliver a metered amount of the drug over a specified period of time.

Of course combinations of drugs and substances in addition to drugs can also be incorporated into the inventive implant device. For example, radioactive tracers such as carbon-14, nonradioactive tracers such as barium sulfate, biocompatibletbiodegradable luminescent compounds, carriers which would transport the drug through skin such as dimethylsulfoxide and dimethylsulfone, water-soluble excipients, etc. could be incorporated with certain drugs for particular applications. The amount of auxiliary agent used will depend, of course, on the specific agent, drug and carrier used to fabricate the implant device as well as the purpose for incorporating the auxiliary agent.

In accordance with a preferred aspect the present invention, there is provided an opiate antagonist implant in the form of a pellet in which the active ingredient antagonist is present in concentrated form as a self-sustaining delivery mechanism for its own dissolution and for delivering an effective amount of an opiate antagonist over a prolonged or extended period of time, preferably inexcess of thirty days and more preferably in excess of sixty and even more preferably in excess of a year. The implant is adapted to be implanted muscularly or subcutaneously or in any effective location.

As discussed in U.S. Pat. No. 6,203,813 it has been found that in some preferred embodiments the use of an anti-inflammatory compound, particularly a steroid. in admixture combination with a pharmaceutical or biologically active substance, or otherwise a particular API, more particularly an opiate antagonist and a pharmaceutical carrier compressed into a pellet provides for unexpectedly long-lasting dosing times, such as, for example, up to approximately eighty days and longer, such as sometimes exceeding six month or longer, thus providing for particularly efficacious drug delivery periods in combination with the engineered polymeric coatings of the invention, for example, the slow-release antagonist delivery for anti-readdiction and/or anti-alcohol abuse therapy, as the case may be.

Any anti-inflammatory agent may be used in this invention, including without limitation, any compound that is effective to reduce blood flow to cellular elements, whether steroidal or non-steroidal, i.e., non-steroidal anti-inflammatory delivery (NSAID). By way of example only, some steroids useful herein include betamethasone dipronionate, betamethasone phosphate, betamethasone valerate, clobetsol propionate, cortisone acetate, dexamethasone phosphate acetate, dexamethasone micronized, fluocinonide, hydrocortisone acetate, hydrocortisone sulfate, methyl prednisone acetate, and trimcinolone acetonide.

The amount of anti-inflammatory compound employed herein may also vary videly depending upon such diverse factors as the specific active ingredient, API or drug employed, the density of the implant, the amount of API to be released in a desired time, the size of the implant and many other factors, all of which are within the realm of consideration of the ordinary skilled practitioner to provide the desirable amount of anti-inflammatory agent for any given situation without undue experimentations. In general, amounts ranging from about ft01% to about 99,9% by wt may be employed.

Any pharmaceutically acceptable carrier or filler may be used in accordance with this invention, including without limitation, magnesium stearate, stearic acid, starch, and cellulose. A preferred carrier is magnesium stearate which is often used as both a lubricant and a binder in tablets, and is particularly preferred because of its decreased solubility in physiological media providing for prolonged implant dissolution and extended drug delivery times, thus enabling the desired therapeutic drug level in a patient's bloodstream for a desired amount of time.

The respective amounts of active ingredient and binder/ anti-inflammatory compound may vary from about 0.01% active ingredient/99.9% binder/anti-inflammatory compound by wt to about 99.99% active ingredient/0.01% binder/anti-inflammatory compound by wt, with the preferred active ingredient range being from about 45% wt to about 95% wt with the remainder carrier or filler material/binder and anti-inflammatory compound.

Further in accordance with the present invention is the preferred effect of using at times, when warranted, depending on, for example, a particular API employed, a compression material or product of an admixture of API, anti-inflammatory agent and pharmaceutically acceptable carrier to form subcutaneously implantable drug delivery devices, e.g., implants, of the invention to be coated with engineered polymeric coatings which are able to dispense therapeutically effective amounts of API over heretofore unknown extended periods of time. Without intending to limit this invention to any particular theory, it is thought that compression rrsed to form, for example, an implantable drug delivery device, provides for a smaller surface area, concomitantly decreasing solubility and thus providing less material per time that can be attacked or he subjected to phagocytosis once implanted. It's also thought that the large size of carrier particles in comparison to the relatively smaller size of drug particles will also provide for longer drug dispersion due to shielding from phagocytosis.

A convenient avenue to more fully illustrate the present invention is to exemplify a preferred aspect of the invention, which is an opiate antagonist implant comprising an admixture of an opiate antagonist API, an anti-inflammatory agent and a pharmaceutically acceptable carrier, and which is prepared by compressing the admixture into a subcutaneously implantable pellet containing antagonist in concentrated form and coated with various ratios of engineered polymeric matrixes as exemplified herein below. While implanted in a patient, the pellet eventually sheds or will lose its polymeric coating or part or all of its encapsulating material, as the case may be, after a time period depending on the particular polymeric ratio employed, and thereafter the API composition starts to dissolve or is permitted to, releasing to a patient over the desired length of time, such as up to and exceeding over a year, therapeutic amounts of opiate/opioid antagonist API effective to block the effects of opiatesiopioids on the human nervous system, and is effective, for example, in inhibiting the effects of endogenous, exogenous, synthetic and natural opiates, and is also effective in inhibiting the effects of a number of other addictive substances including cocaine, alcohol and nicotine.

The respective amounts of API in a depot product may vary from about 0.01% API to 99.9% API by wt, with the preferred API range being from about 45% wt to about 95% wt with the remainder coated polymeric material.

Further in accordance with the present invention is the unexpected and surprising effect of coating on API with engineered time release polymeric coatings to form the subcutaneously implantable depot drug delivery devices, e.g., implants, of the invention which are able to dispense therapeutically effective amounts of active ingredient over heretofore unknown extended periods of time. As shown, the presently inventive implants advantageously avoid the use of conventional complex copolymer delivery systems, polymer matrices, and other expensive and complex time-release agents, which have been found to produce unacceptable complications in humans in earlier studies, along with relatively short dosage release times. The inventive implants are easily manufactured with active ingredient in concentrated for direct subcutaneous implantation in humans to treat a great variety of maladies.

A variety of opiate antagonists can be utilized in the implants of this invention. any of which is not critical to the practice of this invention. Representative examples of such opiate/opioid antagonists include, but are not limited to, naltrexone. naloxone, cyclazocine, diprenorphine, metazocine, levallorphan, rnetaz.ocine, nalorphine, naimefene, and salts thereof, including any pharmaceutically active metabolites. A preferred opiate antagonist is naltrexone, which has received FDA approval for use in humans and has been shown to be free of severe side-effects. Naltrexone is neither addicting nor habit-forming.

Other API opiates and/or opioid substances are also contemplated for use herein, including, but not limited to, for example, buprenorphine (an active ingredient in Suboxone and Subutex) which is known to suppress cravings and withdrawal symptoms and thought to enable patients to make long-term behavior changes to ultimately achieve sustained addiction remission, and Tramadol used in some accelerated opiate/opioid withdrawal programs. Still other API substances used in opioid and opiate withdrawal and other withdrawal methods, such as Clonidine, are contemplated for use herein and other withdrawal substances such as Nalmafene used for treatment of alcoholism as discussed, for example, in Costantini, “Implantable Technology for long-term delivery of Nalmefene for treatment of alcoholism” Int'l J. of Pharmaceutics, 283 (2004) 34-35, the disclosure of which is incorporated by reference in its entirety are also contemplated for use herein.

It is to be understood however, that the following examples are for illustrative purposes only and are not intended to limit the scope of this invention or claims in any ay.

### EXAMPLE 1

### Pgla Coated Naltrexone Depot Implant

Naltrexone pellets with a PGLA coating ratio of 50:50, a coating ratio of 65:35, a coating ratio of 75:25 and a coating ratio of 85:15; or other APis coated with such engineered amorphous polymeric coatings may be prepared as follows:

### Coating Preparation

A desired amount of PLA/PGA resin with a combination of 65% PLA and 35% PGA of USP grade resin suitable for pharmaceutical usage is provided. A desired amount of preferably USP grade or pharmaceutical grade THF or methylene chloride solvent, or other suitable solvent, is then combined with the PLA/PGA resin mixture previously prepared, and placed into a mixing environment, such as a mechanical vibrator for mixing. This procedure may be repeated, such as, for example, for 75% PLA/25% PLEA resin and 85% PLA/15% PGA resins mixtures with the same solvents THF or methylene chloride, or any other suitable solvent mixed in the same manner.

### Coating Procedure

The thus mixed PLA/PGA solvent is then preferably placed in a receptacle, such as into a wide mouth stainless steel dish or similar device. Drug pellets such as prepared in accordance with U.S. 6,203,813 are put into the solvent mixture and coated entirely and then preferably placed on a non-stick surface, such as a Teflon sheet, to prevent the pellets from sticking after drying, Excess solvent may be discarded or otherwise removed.

After the finished the coated pellets are thus prepared, they are dried and/or excess solvent further removed, such as by placing into a heated drying environment, such as an air-circulated oven, The oven temperature can be, for example, at 35-40° C.

The pellets are ready to then be packaged for use,

### Plastic Syringe Preparation

Coated pellets may be then put into a syringe body or syringe device in one example of depot insertion into a patient,

In use, preferably a first stage depot uncoated API-comprising pellet is deployed, such as in a muscular area, or subcutaneously, and will last approximately 2 months, followed by a second coated pellet prepared in the above manner which will lose its coating and last an additional two months, followed by a third coated pellet which will lose its coating and last the next two months, and finally followed by a fourth coated pellet which will lose its coating and last the next two months. It is contemplated that there will be some overlap such that the preparation of the four coated pellets in this example will be completely used in an approximate 5-7 month period for an average 6 month duration of medication administration, starting with a first depot implanted uncoated API-comprising pellet,

It will be appreciated that as an added advantage of the present invention, as used as coating materials, such as certain ratios of PLA/PGA copolymeric coatings, much less polymer materials are used in the inventive sustained release/ controlled release API delivery systems as compared to conventional microsphere preperataions, which is advantageous in the cost of preparation and in use, for example, in eliminating, or at least substantially reducing metabolic acidosis associated with preparations requiring larger amounts or quantities of polymeric materials. Additionally, the polymer coating or shell prevents breakage that would lead to more surface exposure of the API, which in turn would lead to more rapid degradations.

Further, in preferred embodiments, the steroid in the preparation is hydrophobic, and helps keep water away from pellets and their coatings and extends the duration of API within its polymeric coating shell.

Additionally, the coating protects the API inside from degradation by phagocytosis because the polymer is resistance to phagocytosis to a much greater extent than the API inside, thought probably due to larger polymer molecules and tighter bonding that resists and possibly prevents phagocytosis until hydration and hydrorlysis of the polymer produces degradation of the coating or shell to expose the API.

### EXAMPLE 2

The following is a further example embodiment of API in premade biodegradable polymer capsules of biodegradable polyesters (PLGA, PLA, PCL, etc.) for generating polymer encapsulated drug pellets with timed release properties.

Materials/Equipment

The list below displays a list of reagents and equipment used in the course of this example.

Poly(lactide-co-glycolide) (PLGA, PolyVivo Akina, Inc.)

Poly(lactide) (PLA, PolyVivo Akina, Inc., Evonik, Corbion, and Durect)

Poly(caprolactone) (PCL, PolyVivo Akina, Inc.)

Compressed Nitrogen/Argon (Indiana Oxygen Company)

Liquid Nitrogen (Indiana Oxygen Company)

Isotemp Vacuum Oven Model 280A (Fisher)

Duoseal Vacuum Pump (Welch model 1350)

Incubator (Barnstead Model 4450SHKE)

Accumet Basic pH Meter (Fisher, AB15)

Texture Analyzer (TA.XTPlus, Texture Technologies Corp)

UV/Vis spectrophotometer (Genesys 6, Thermo-Fisher)

Airbrush System (Aztek, A7778)

Air compressor (Craftsman, 3 Gal)

Stereoscope (Navitar)

Microscope (Olympus BX51)

Digital Camera (Olympus DP70)

Image Analysis Software (Olympus, Microsuite 5)

Gelatin Capsules, size 13 (Election Microscopy Sciences)

Loctite All Purpose Go2 Glue

Weller Soldering Iron

Poly(Vinyl alcohol) (146,000Da, Aldrich) (PVA)

Dichloromethane (DCM)

Acetonitrile (ACN)

Acetone (ACE)

ethyl acetate (ETAC)

Tetrahydrofuran(THF)

Toluene (TOL)

Xylene (XYL)

Ethanol (ETOH)

Distilled water (H2O)

NOW Healthy Food Triple “000” Gelatin Caps 200 empty capsules

Methods/Observations

All work performed using 0.40 Naltrexone comprising “NTX” pellet samples composed in accordance with methods described in U.S. Pat. No. 6,203,813, placing pellets inside portions of capsules manufactured from bio-degradable polymer and sealing the two sides of the capsule to enclose the pellet.

Polymer chart

As several polymers are used in this example, such are referenced in a short description for each catalog number polymer below. Below is a breakdown of the polymer number and its respective description (Table 1).

TABLE 1
Polymer description breakdown.

| Catalog # | Sample Name | Polymer Description |
|---|---|---|
| AP001 | PDLA-E-10K | P(D,L)LA ester end cap (Mₙ 10 kDa-15 kDa) |
| AP003 | PDLA-E-45K | P(D,L)LA ester end cap (Mₙ 45 kDa-50 kDa) |
| AP004 | PDLA-E-65K | P(D,L)LA ester end cap (65 kDa-75 kDa) |
| AP006 | PLLA-A-100K | P(L)LA acid end cap (Mₙ 100 kDa-125 kDa) |
| AP007 | PLLA-A-125K | P(L)LA acid end cap (Mₙ 125 kDa-150 kDa) |
| AP008 | PLLA-A-400K | P(L)LA acid end cap (Mₙ 400 kDa-500 kDa) |
| AP017 | PLGA-A-60L-75K | PLGA 60:40 acid end cap (Mₙ 75 kDa-85 kDa) |
| AP018 | PLGA-A-75L-45K | PLGA 75:25 acid end cap (Mₙ 45 kDa-55 kDa) |
| AP020 | PLGA-A-85L-100K | PLGA 85:15 acid end cap (Mₙ 100 kDa-200 kDa) |
| AP024 | PLGA-E-85L-35K | PLGA 85:15 ester end cap (Mₙ 35 kDa-45 kDa) |
| AP035 | PDLA-A-55K | P(D)LA acid end cap (Mₙ 55 kDa-56 kDa) |
| AP043 | PLGA-A-60L-45K | PLGA 60:40 acid end cap (Mₙ 45-55 kDa) |
| AP047 | PLLA-A-45K | P(L)LA acid end cap (Mₙ 45 kDa-55 kDa) |
| AP049 | PLGA-A-90L-100K | PLGA 90:10 acid end cap (100-200 kDa) |
| AP050 | PLLA-A-300K | P(L)LA acid end cap (Mₙ 300 kDa-400 kDa) |
| AP054 | PLGA-E-75L-25K | PLGA 75:25 ester end cap (Mₙ 25 kDa-35 kDa) |
| AP063 | PLGA-E-50L-15K | PLGA 50:50 ester end cap (Mₙ 15 kDa-25 kDa) |
| AP065 | PLLA-A-150K | P(L)LA acid end cap (Mₙ 150 kDa-200 kDa) |
| AP072 | PDLA-E-85K | P(D)LA ester end cap (Mₙ 85 kDa-95 kDa) |
| AP079 | PLLA-E-5K | P(L)LA ester end cap (Mₙ 5 kDa-10 kDa) |
| AP085 | PLGA-E-90L-25K | PLGA 90:10 ester end cap (Mₙ 25 kDa-35 kDa) |
| AP087 | PLGA-A-85L-45K | PLGA 85:15 acid end cap (Mₙ 45-55 kDa) |
| AP097 | PLGA-C-50L-35K | PLGA 50:50 cholesterol end cap (Mₙ 35 kDa-45 kDa) |
| AP098 | PLGA-C-50L-15K | PLGA 50:50 cholesterol end cap (Mₙ 15 kDa-25 kDa) |
| AP099 | PLGA-C-85L-35K | PLGA 85:15 cholesterol end cap (Mₙ 35 kDa-45 kDa) |
| AP100 | PLGA-C-85L-15K | PLGA 85:15 cholesterol end cap (Mₙ 15 kDa-25 kDa) |
| AP106 | PLGA-A-90L-55K | PLGA 90:10 acid end cap (Mₙ 55 kDa-65 kDa) |
| AP114 | PDLA-A-85K | P(D,L)LA acid end cap (Mₙ 85 kDa-100 kDa) |
| AP119 | PLGA-E-90L-100K | PLGA 90:10 ester end cap (Mₙ 100 kDa-200 kDa) |
| AP121 | PLGA-E-50L-45K | PLGA 50:50 ester end cap (Mₙ 45 kDa-50 kDa) |
| AP122 | PLGA-A-90L-55K | PLGA 90:10 acid end cap (Mₙ 55 kDa-85 kDa) |
| AP128 | PLLA-A-15K | P(L)LA acid end cap (Mₙ 15 kDa-25 kDa) |

Film Longevity

0.5 cm squares of various polymer films were cut out and placed in a 12 well plate. Each well was then filled with DIH2O and placed in a 37C incubator. Periodically they were pictured to observe any degradation.

Capsule Filling

Gelatin capsules were utilized to make more rounded films for the film coating process. Solutions of 1%, 5%, 10% and 20% of PLA or PLGA, respectively, were made in DCM and then were used to fill each half of an empty capsule. Capsules were sized to a similar diameter of the NTX pellet. This was allowed to dry overnight and then placed in DIH2O to dissolve or swell the gelatin and release the polymer films from the capsules. These polymer films were then allowed to dry overnight to get rid of excess water. The polymer capsules were then trimmed down to make two bowl shape films. These bowl shaped films were then used to encapsulate a NTX pellet and then, using various glues, the seams were sealed. These samples were then allowed to dry out overnight to set the glue. Once sealed the encapsulated pellets were placed in 10mL of DIH2O and placed in a 37 ° C./100RPM shaking incubator and observed until red dye leaked out.

Polymer Glue

Various polymer glues may be used to seal API comprising pellets in accordance with the invention, such as NTX pellets in this example. In one preferred embodiment this may be done by weighing out, for instance, a half gram of various PLGA polymers and adding 2 or 3 mL of DCM to dissolve, then allowing the excess DCM to evaporate until the viscosity of the polymer is desirably thick and stringy as possible. Bowl shaped films may then be wrapped around the pellet, and a pipette tip may be used to scoop out some of the glue and then painted over the seam to make a seal.

Coating observation

Coating observations were performed using Navitar stereoscope between 0.7-4X. Typically observations were performed under normal lighting conditions.

Incubation

For time release testing, the pellets were incubated with 10m1 of water in scintillation vials and observed periodically to determine dye loss by observing a pink to red color develop within the water.

2-Month Commercial Polymer Products Wrapping

For a 2-month formulation a preferred product has been found to be AP114. For scale-up, it is contemplated to utilize a large-scale commercial PLA from a GMP environment. Three commercial GMP manufactured products were used to determine the use of similar products in the same manner as the Polyvivo AP114 polymer used in the preferred 2-month pellet wrapping. These products were selected to be PLA's with similar characterstics to AP114. These include Pursorb (Corbion), R207S (Evonik), and Lactel (Cat# B6005-2). These were made using the same method as stated in previous updates. For each of the products three red NTX pellets were wrapped and placed in approximately 10 mL of water then placed in a 37C shaking incubator and monitored for color change in the water.

A preferred example embodiment herein is Evonik R207S.

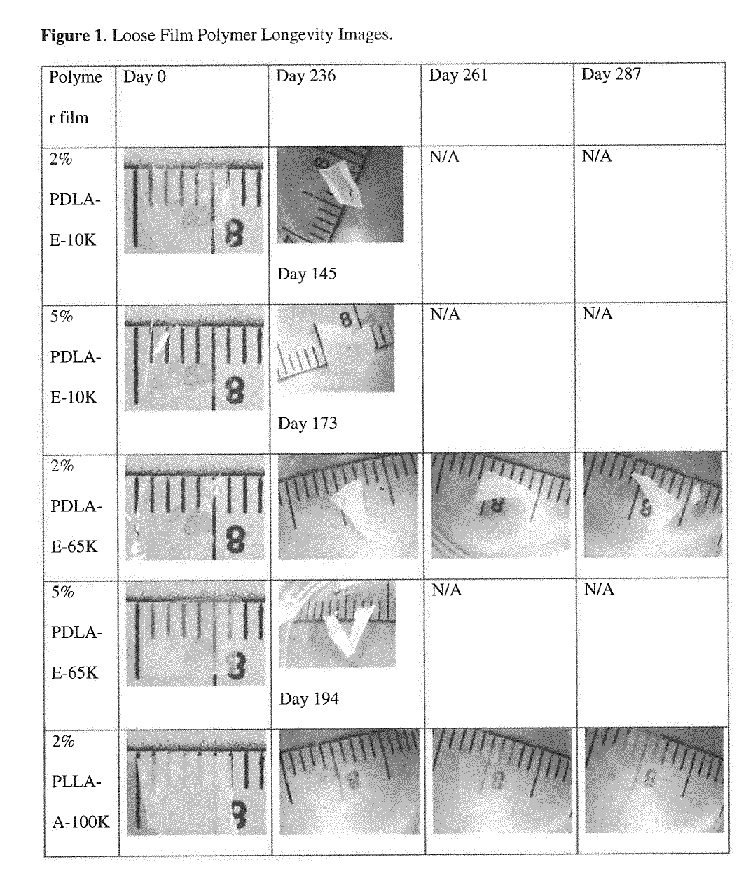

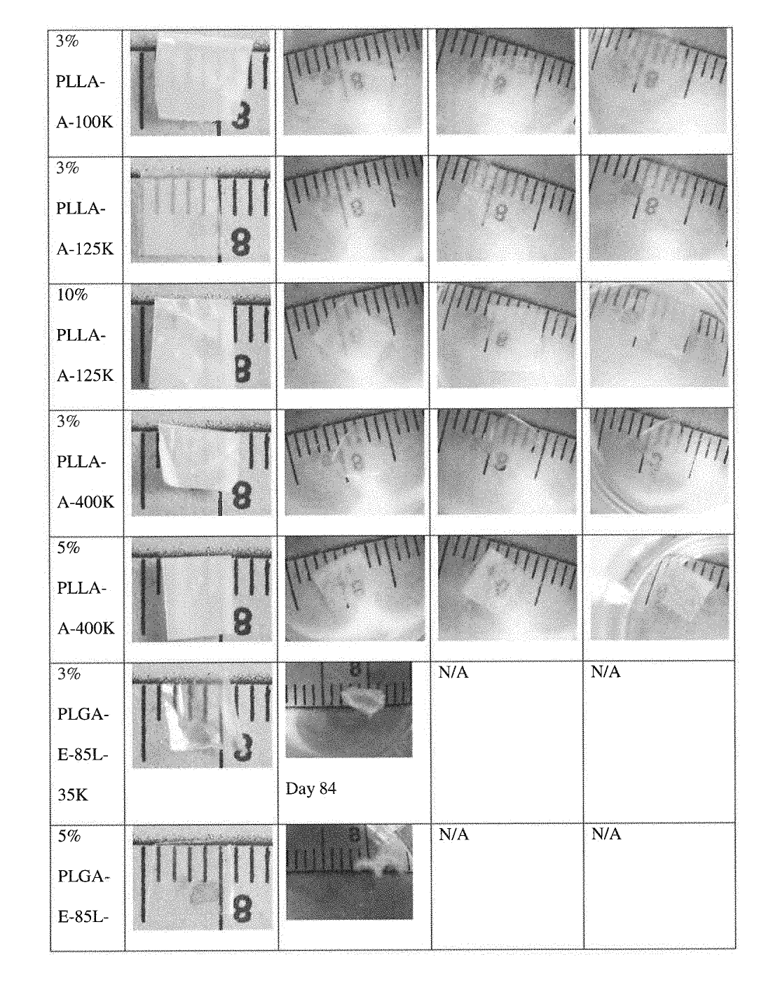

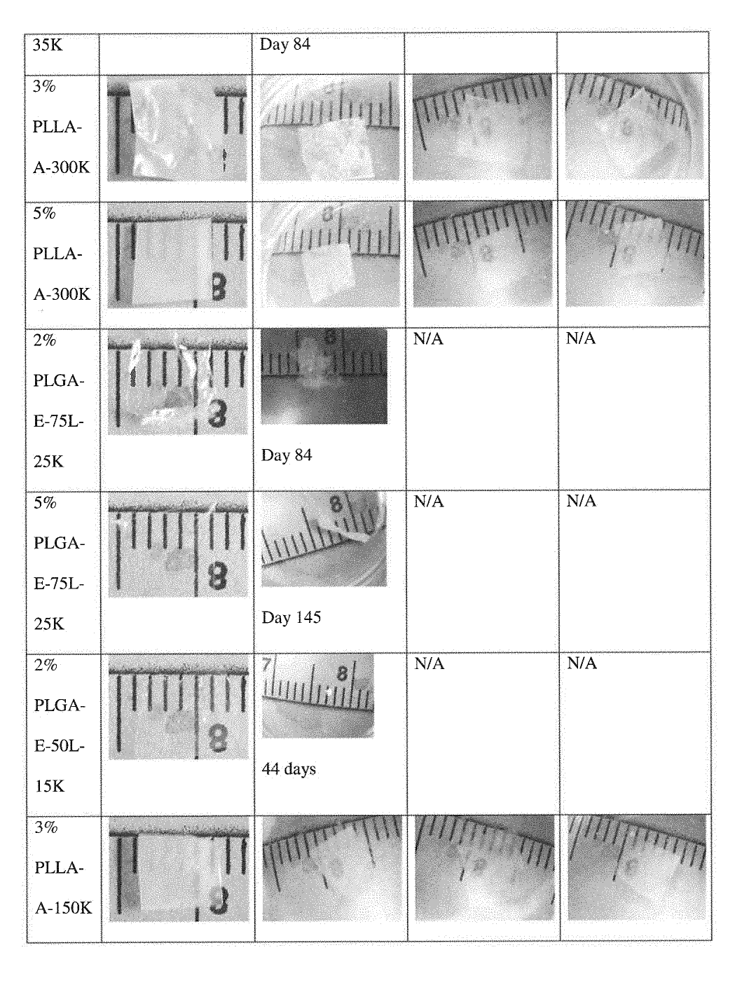

Coating observations are as indicated for each pellet type below with images and description shown respectively. All tests performed with incubation at 37° C./100RPM unless otherwise specified. Unless otherwise specified images were collected at 0.7X magnification under normal lighting. The scale bar in the background has hash-marks at 1mm intervals. FIG. 1 shows results from loose film incubation tests. When degradation was observed pictures were no longer continued.

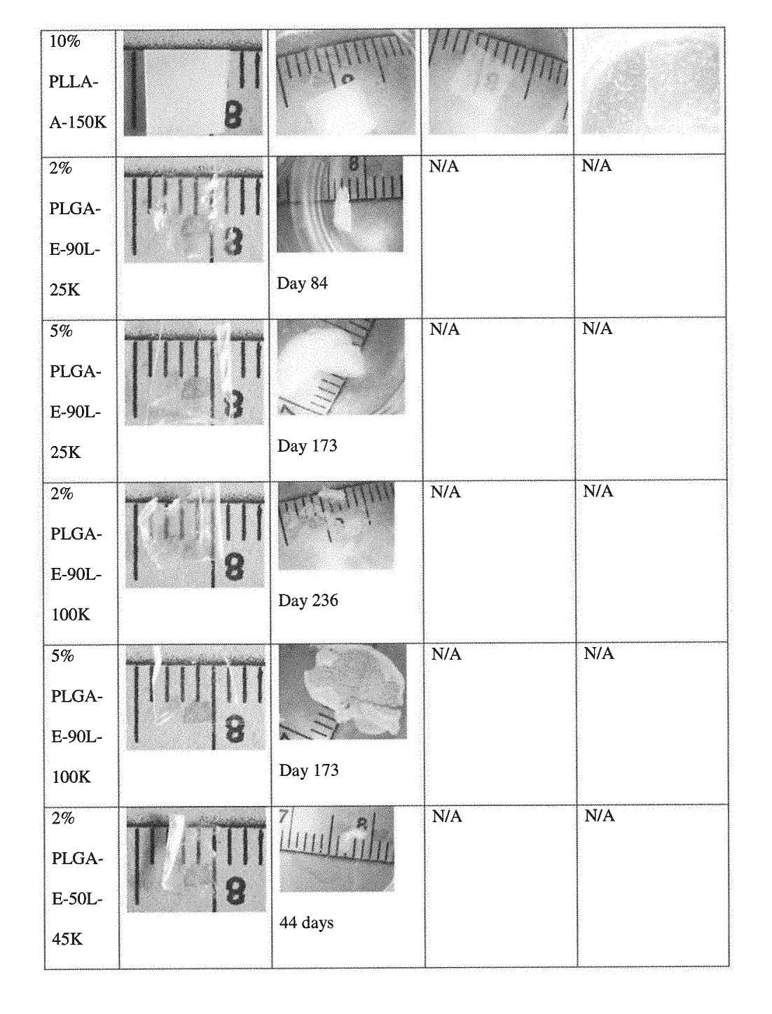

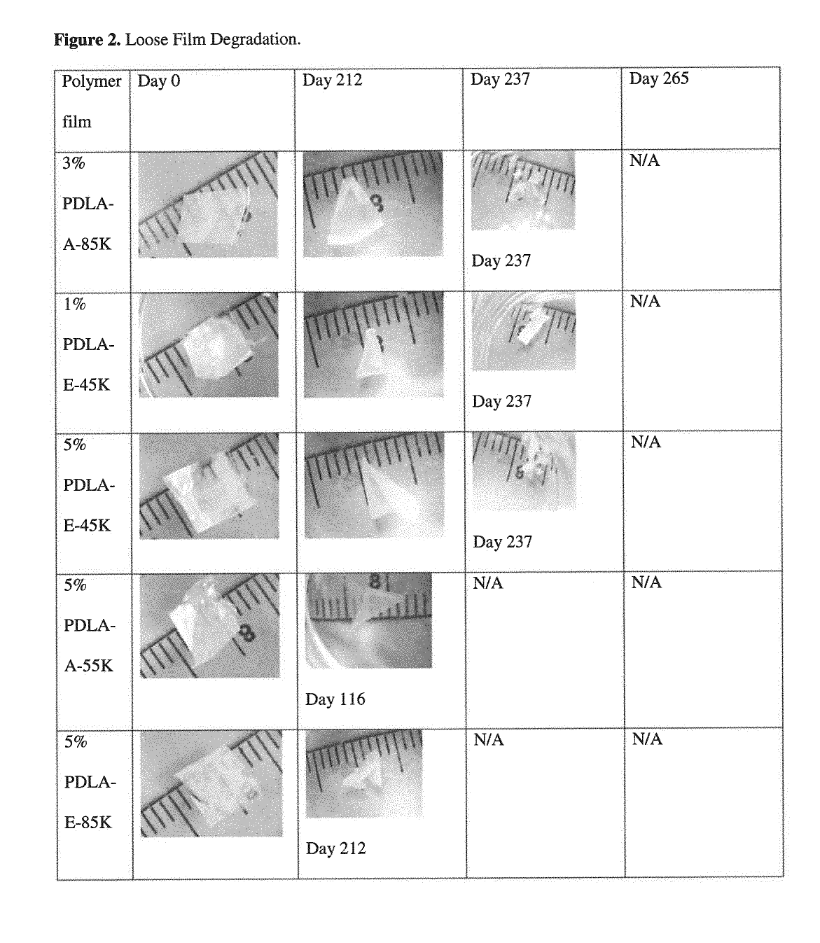

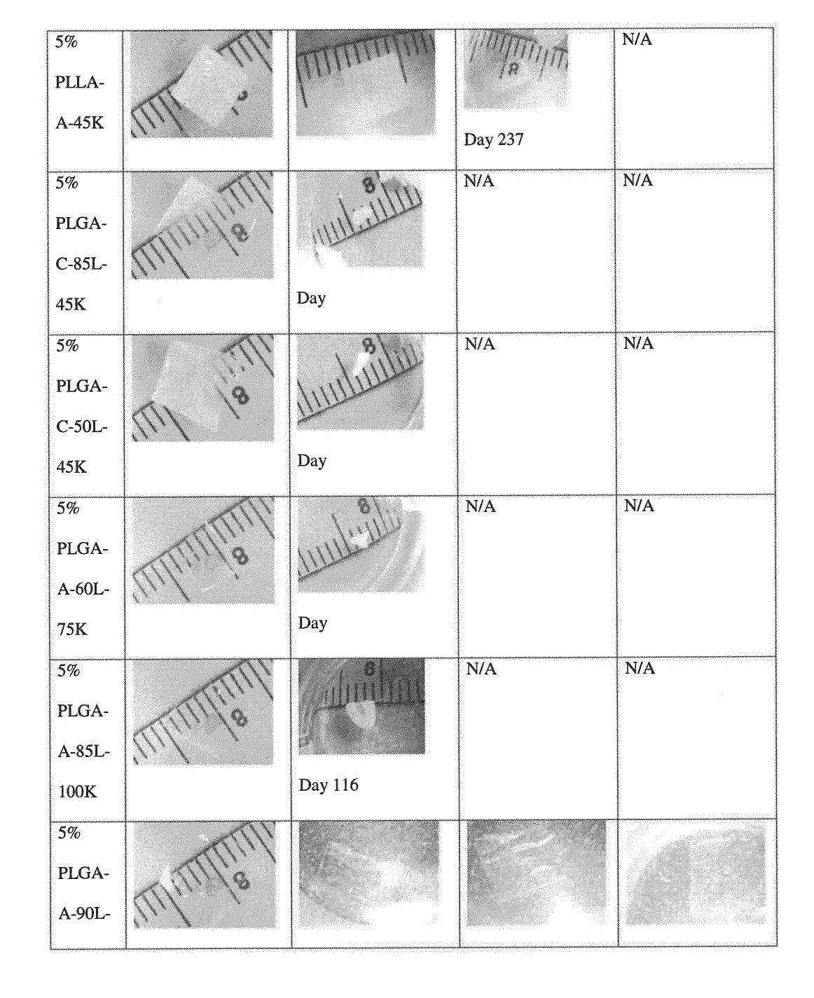

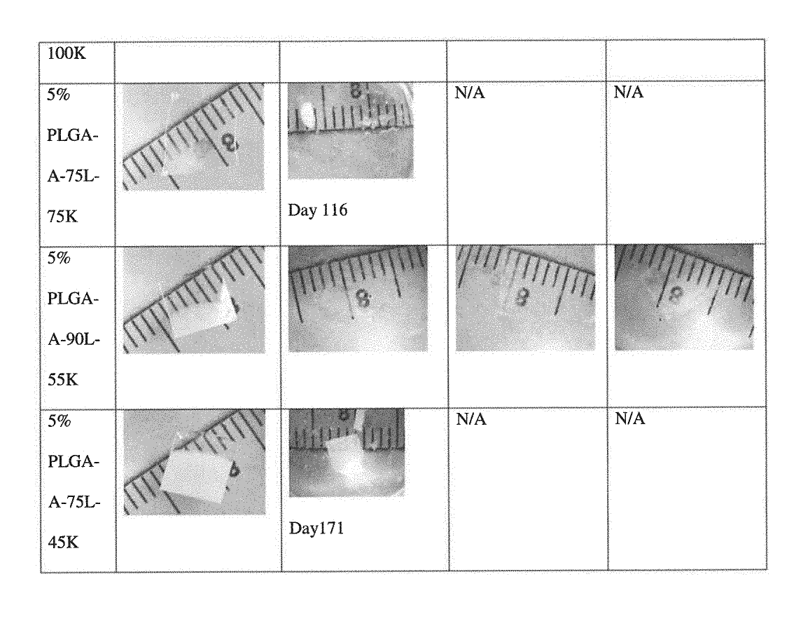

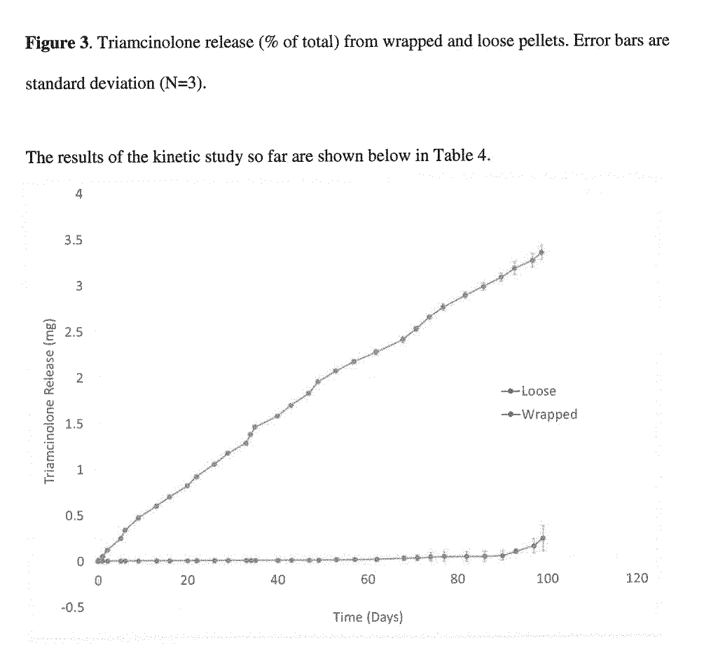

Another series of polymer films examples were started and tested by the same method, with representative pictures shown below (FIG. 2).

Four and Six Month Tests

Reproducibility tests on four and six month formulations have been initiated for polymers which had results indicating their applicability to these time-lines in screening tests. These include triple wrapped AP004 and AP058 glued with AP003 for four-month formulations as well as AP138, AP119, and AP020 for six-month formulations.

### HPLC Assay Quantitative Kinetic Study

Assay Method

Triamcinolone was assayed on the previously detailed Shimadzu HPLC system equipped with a Synergi 4u Fusion 150×4.6mm 80A column. The mobile phase was an eluent of 1:1 water to acetonitrile flowing at 1 mL/min. Detection was done at a wavelength of 242nm and the triamcinolone acetonide peak was detected at 3.3 minutes during 10 minute runs.

Standards were made by serially dilution of triamcinolone acetonide in mobile phase. Standards were made by serially dilution of provided triamcinolone acetonide in mobile phase.

Study Method

A kinetic study was initiated to test for the release from the pellets. Unstained pellets were used for the drug kinetic assay. For this test, three pellets were wrapped with the previously found to be effective for preventing dye-leakage for up to two months. This wrapping was comprised of shells made using 10% w/v PDLA-A-85K (AP114) glued to each other using a 60% w/v solution of PDLA-A-85K (AP114) in dichloromethane. Three, unwrapped pellets were used as a control series.

All samples were incubated in 50m1 centrifuge tubes with 10m1 of PBST with 0.1% sodium azide. At each predetermined time interval, a sample was taken from the tube for HPLC analysis and the rest of the solution was discarded and replaced with fresh PBST.

Results

In addition to the 2-month formulation which has been identified. Other formulations have been found during the dye-release test which show failure at the selected time points as follows (Table 2):

| Time line | Formulation description |
|---|---|
| 4 month | 1% AP004x3; AP003 glue |
| formulations | 1% AP050x3; AP003 glue |
| | 5% AP085; AP006 glue |
| | 5% AP085; AP003 glue |
| | 5% AP085; AP072 glue |
| | 5% AP085; AP079 glue |
| | 5% AP024; AP072 glue |
| | 5% AP024; AP003 glue |
| | 5% AP024; AP006 glue |
| | 5% AP035; AP099 glue |
| 6 month | 5% AP119; AP119 |
| formulations | 5% AP003; AP035 |

Additionally, GMP grade Evonik R207S is contemplated for use in preparation of drug loaded test samples for in-vitro release assay.

### EXAMPLE 3

This example is a coating application embodiment of biodegradable polyesters (PLGA, PLA, PCL, etc.) to generate polymer coated drug pellets with timed release properties.

Materials/Equipment

The list below displays a list of reagents and equipment used in the course of this example.

Poly(lactide-co-glycolide) (PLGA, PolyVivo Akina, Inc.)

Poly(lactide) (PLA, PolyVivo Akina, Inc.)

Poly(caprolactone) (PCL, PolyVivo Akina, Inc.)

Compressed Nitrogen/Argon (Indiana Oxygen Company)

Liquid Nitrogen (Indiana Oxygen Company)

Isotemp Vacuum Oven Model 280A (Fisher)

Duoseal Vacuum Pump (Welch model 1350)

Incubator (Barnstead Model 4450SHKE)

Accumet Basic pH Meter (Fisher, AB15)

Texture Analyzer (TA.XTPlus, Texture Technologies Corp)

UV/Vis spectrophotometer (Genesys 6, Thermo-Fisher)

Airbrush System (Aztek, A7778)

Air compressor (Craftsman, 3 Gal)

Stereoscope (Navitar)

Microscope (Olympus BX51)

Digital Camera (Olympus DP70)

Image Analysis Software (Olympus, Microsuite 5)

Gelatin Capsules, size 13 (Election Microscopy Sciences)

Loctite All Purpose Go2 Glue

Weller Soldering Iron

Poly(Vinyl alcohol) (146,000Da, Aldrich) (PVA)

Dichloromethane (DCM)

Acetonitrile (ACN)

Acetone (ACE)

Ethyl acetate (ETAC)

Tetrahydrofuran(THF)

Toluene (TOL)

Xylene (XYL)

Ethanol (ETOH)

Distilled water (H2O)

NOW Healthy Food Triple “000” Gelatin Caps 200 empty capsules

Methods/Observations

All work was performed using 0.40 Natrexone comprising “NTX” pellet samples composed in accordance with methods described in US Patent No. 6,203,813, placing pellets inside portions of capsules manufactured from biodegradable polymers and sealing the two sides of the capsule to enclose the pellet. Drug kinetics work was performed using 0.40 NTX unstained pellets.

Polymer chart

Below is a breakdown of the polymer number and its respective description (Table 3).

TABLE 3
Polymer description breakdown.

| Catalog # | Sample Name | Polymer Description |
|---|---|---|
| AP001 | PDLA-E-10K | P(D,L)LA ester end cap (Mn 10 kDa-15 kDa) |
| AP003 | PDLA-E-45K | P(D,L)LA ester end cap (Mn 45 kDa-50 kDa) |
| AP004 | PDLA-E-65K | P(D,L)LA ester end cap (65 kDa-75 kDa) |
| AP006 | PLLA-A-100K | P(L)LA acid end cap (Mn 100 kDa-125 kDa) |
| AP007 | PLLA-A-125K | P(L)LA acid end cap (Mn 125 kDa-150 kDa) |
| AP008 | PLLA-A-400K | P(L)LA acid end cap (Mn 400 kDa-500 kDa) |
| AP017 | PLGA-A-60L-75K | PLGA 60:40 acid end cap (Mn 75 kDa-85 kDa) |
| AP018 | PLGA-A-75L-45K | PLGA 75:25 acid end cap (Mn 45 kDa-55 kDa) |
| AP020 | PLGA-A-85L-100K | PLGA 85:15 acid end cap (Mn 100 kDa-200 kDa) |
| AP024 | PLGA-E-85L-35K | PLGA 85:15 ester end cap (Mn 35 kDa-45 kDa) |
| AP035 | PDLA-A-55K | P(D)LA acid end cap (Mn 55 kDa-56 kDa) |
| AP043 | PLGA-A-60L-45K | PLGA 60:40 acid end cap (Mn 45-55 kDa) |
| AP047 | PLLA-A-45K | P(L)LA acid end cap (Mn 45 kDa-55 kDa) |
| AP049 | PLGA-A-90L-100K | PLGA 90:10 acid end cap (100-200 kDa) |
| AP050 | PLLA-A-300K | P(L)LA acid end cap (Mn 300 kDa-400 kDa) |
| AP054 | PLGA-E-75L-25K | PLGA 75:25 ester end cap (Mn 25 kDa-35 kDa) |
| AP063 | PLGA-E-50L-15K | PLGA 50:50 ester end cap (Mn 15 kDa-25 kDa) |
| AP065 | PLLA-A-150K | P(L)LA acid end cap (Mn 150 kDa-200 kDa) |
| AP072 | PDLA-E-85K | P(D)LA ester end cap (Mn 85 kDa-95 kDa) |
| AP079 | PLLA-E-5K | P(L)LA ester end cap (Mn 5 kDa-10 kDa) |
| AP085 | PLGA-E-90L-25K | PLGA 90:10 ester end cap (Mn 25 kDa-35 kDa) |
| AP087 | PLGA-A-85L-45K | PLGA 85:15 acid end cap (Mn 45-55 kDa) |
| AP097 | PLGA-C-50L-35K | PLGA 50:50 cholesterol end cap (Mn 35 kDa-45 kDa) |
| AP098 | PLGA-C-50L-15K | PLGA 50:50 cholesterol end cap (Mn 15 kDa-25 kDa) |
| AP099 | PLGA-C-85L-35K | PLGA 85:15 cholesterol end cap (Mn 35 kDa-45 kDa) |
| AP100 | PLGA-C-85L-15K | PLGA 85:15 cholesterol end cap (Mn 15 kDa-25 kDa) |
| AP106 | PLGA-A-90L-55K | PLGA 90:10 acid end cap (Mn 55 kDa-65 kDa) |
| AP114 | PDLA-A-85K | P(D,L)LA acid end cap (Mn 85 kDa-100 kDa) |
| AP119 | PLGA-E-90L-100K | PLGA 90:10 ester end cap (Mn 100 kDa-200 kDa) |
| AP121 | PLGA-E-50L-45K | PLGA 50:50 ester end cap (Mn 45 kDa-50 kDa) |
| AP122 | PLGA-A-90L-55K | PLGA 90:10 acid end cap (Mn 55 kDa-85 kDa) |
| AP128 | PLLA-A-15K | P(L)LA acid end cap (Mn 15 kDa-25 kDa) |

Film Longevity

0.5 cm squares of various polymer films were cut out and placed in a 12 well plate. Each well was then filled with DIH2O and placed in a 37C incubator. Periodically they were pictured to observe any degradation.

Capsule Filling

Gelatin capsules were utilized to make more rounded films for the film coating process. Solutions of 1%, 5%, 10% and 20% of PLA or PLGA, respectively, were made in DCM and then were used to fill each half of an empty capsule sized to a similar diameter which is similar to the NTX pellet. This was allowed to dry overnight and then placed in DIH2O to dissolve or swell the gelatin and release the polymer films from the capsules. These polymer films were then allowed to dry overnight to get rid of excess water. The polymer capsules were then trimmed down to make two bowl shape films. These bowl shaped films were then used to encapsulate a NTX pellet and then, using various glues, the seams were sealed. These samples were then allowed to dry out overnight to set the glue. Once sealed the encapsulated pellets were placed in 10mL of DIH2O and placed in a 37 ° C./100RPM shaking incubator and observed until red dye leaked out.

Polymer glue

Various polymer glues were made to seal the NTX pellets This was done by weighing out a half gram of various PLGA polymers and adding 2 or 3 mL of DCM to dissolve, then allowing the excess DCM to evaporate until the viscosity of the polymer was desirably thick and stringy. Once the bowl shaped films were wrapped around the pellet a pipette tip was used to scoop out some of the glue and then painted over the seam to make a seal.

Coating observation

Coating observations were performed using Navitar stereoscope between 0.7-4X. Typically observations were performed under normal lighting conditions.

Incubation

For the preliminary test the pellets were incubated with 10m1 of water in scintillation vials and observed periodically to determine dye loss by observing a pink to red color develop within the water.

Results/Discussion

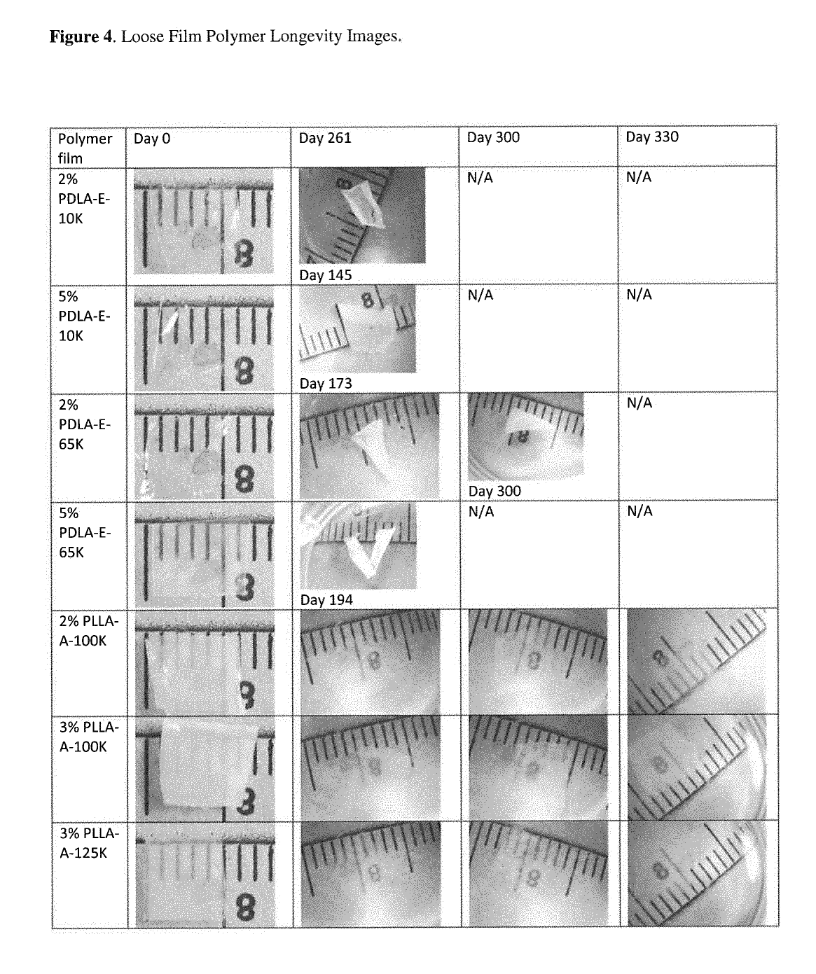

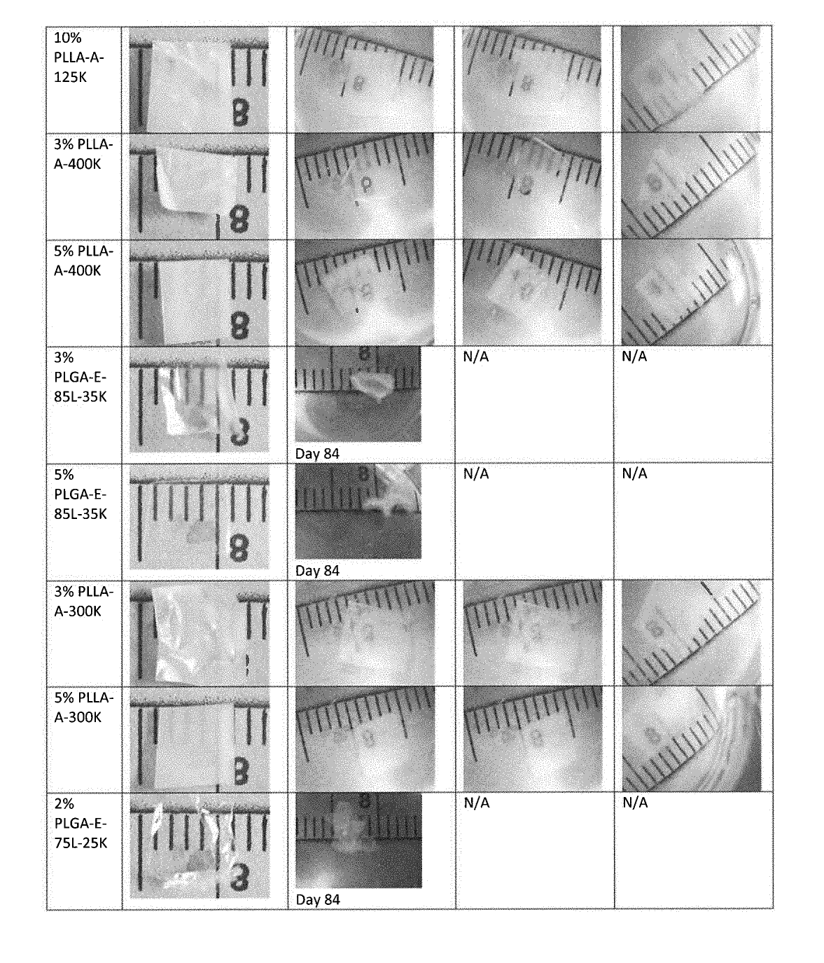

Coating observations are as indicated for each pellet type below with images and description shown respectively. All tests performed with incubation at 37° C./100RPM unless otherwise specified. Unless otherwise specified images were collected at 0.7X magnification under normal lighting. The scale bar in the background has hash-marks at 1mm intervals. FIG. 4 shows results from loose film incubation tests. When degradation was observed pictures were no longer continued.

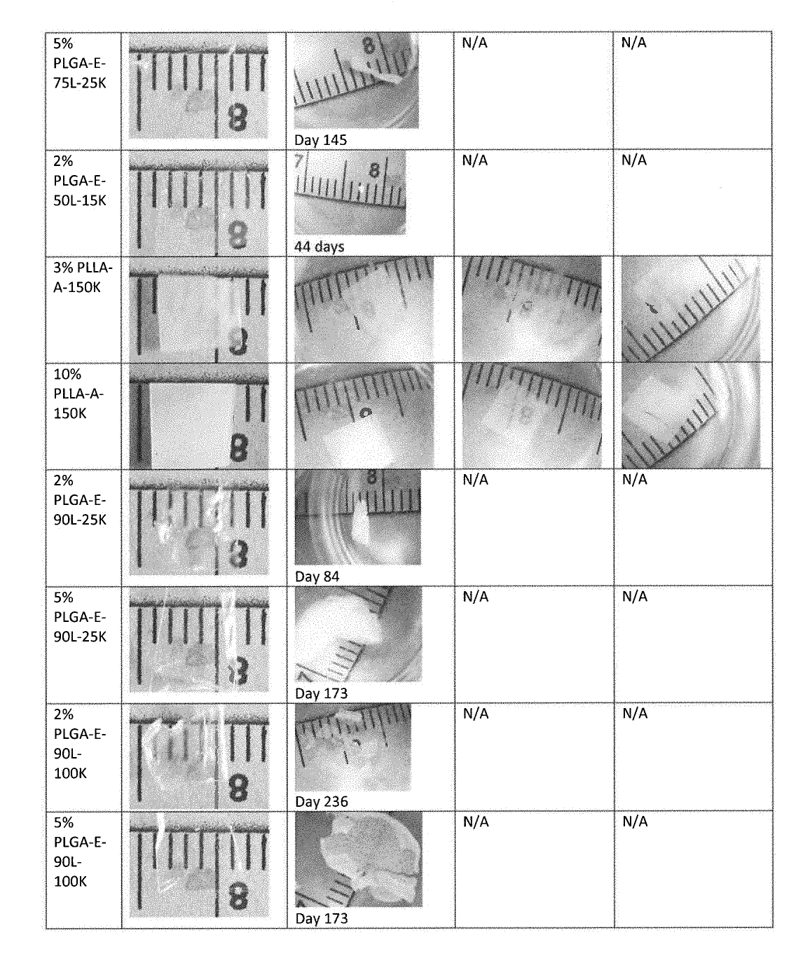

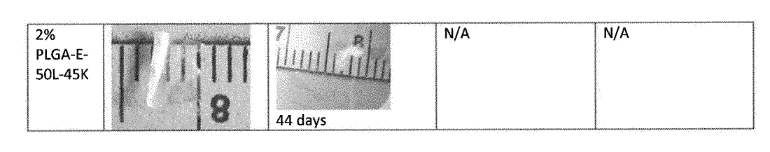

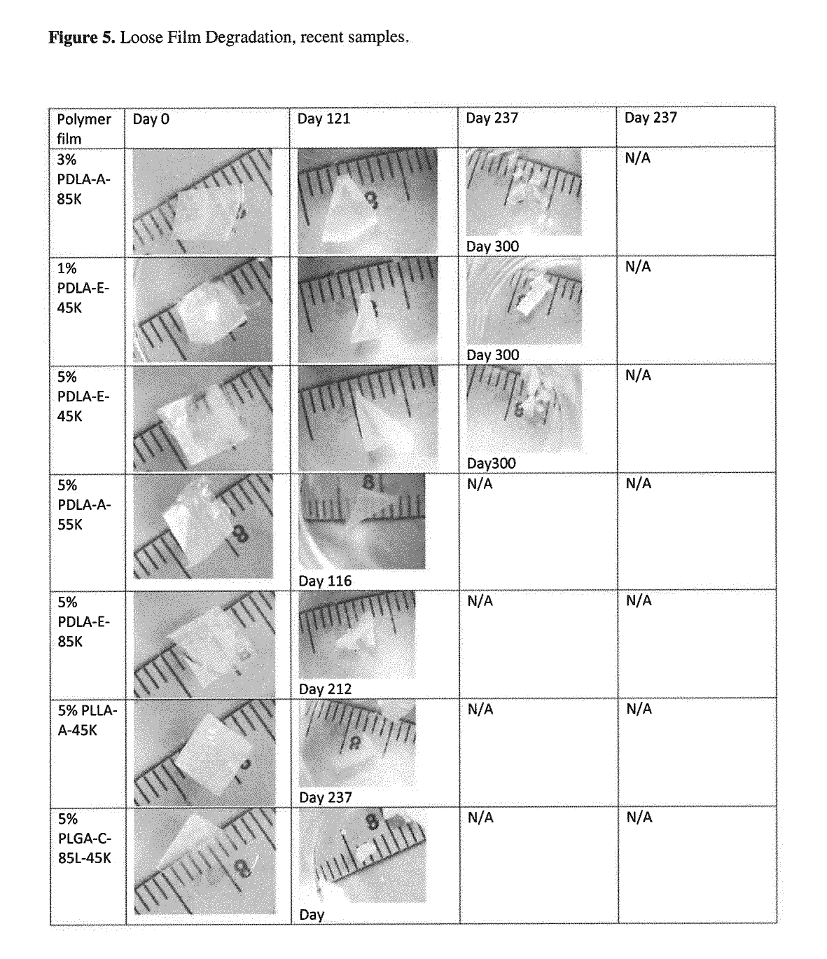

Another series of polymer films were started and tested by the same method. Their pictures are shown below (FIG. 5).

Incubation Tests

Are listed in Table 4.

TABLE 4
Incubation Tests

| | Sample | | |
|---|---|---|---|
| | Blank (control) | AP114 coated | R207S coated |
| Time (days) | mg triamcinolone released (average N = 3) | | |
| 1 | 0.051211485 | 0 | 0 |
| 2 | 0.120648829 | 0 | 0 |
| 5 | 0.24384248 | 0 | 0 |
| 6 | 0.336319817 | 0 | 0 |
| 9 | 0.470401456 | 0 | |
| 13 | 0.597032413 | 0 | |
| 16 | 0.697787328 | 0 | |
| 20 | 0.816961448 | 0 | |
| 22 | 0.91586195 | 0 | |
| 26 | 1.051420005 | 0 | |
| 29 | 1.170646126 | 0 | |
| 33 | 1.279243192 | 0 | |
| 34 | 1.372989231 | 0 | |
| 35 | 1.456256948 | 0 | |
| 40 | 1.572306905 | 0 | |
| 43 | 1.690557997 | 0 | |
| 47 | 1.817966604 | 0 | |
| 49 | 1.945492587 | 0 | |
| 53 | 2.059960999 | 0.00294831 | |
| 57 | 2.162032964 | 0.00294831 | |
| 62 | 2.2648377 | 0.004101083 | |
| 68 | 2.39868526 | 0.014231697 | |
| 71 | 2.517308209 | 0.018141038 | |
| 74 | 2.643061451 | 0.026493128 | |
| 77 | 2.748299648 | 0.030764462 | |
| 82 | 2.879239432 | 0.030764462 | |
| 86 | 2.972327181 | 0.03225592 | |
| 90 | 3.072392391 | 0.039143235 | |
| 93 | 3.170684343 | 0.085801617 | |
| 97 | 3.25367618 | 0.146171862 | |
| 99 | 3.340624669 | 0.226956811 | |
| 105 | 3.447484905 | 0.341229652 | |
| 112 | 3.55907688 | 0.461298995 | |
| 117 | 3.654480766 | 0.596547467 | |
| 121 | 3.739091528 | 0.736299738 | |
| 125 | 3.827940227 | 0.862098456 | |
| 127 | 3.904710948 | 0.984996809 | |
| 131 | 3.981777605 | 1.106551569 | |

### HPLC Assay Quantitative Kinetic Study

Assay Method

Triamcinolone was assayed on the previously detailed Shimadzu HPLC system equipped with a Synergi 4u Fusion 150×4.6mm 80A column. The mobile phase was an eluent of 1:1 water to acetonitrile flowing at 1 mL/min. Detection was done at a wavelength of 242nm and the triamcinolone acetonide peak was detected at 3.3 minutes during 10 minute runs.

Standards were made by serially dilution of customer provided triamcinolone acetonide in mobile phase. Standards were made by serially dilution of provided triamcinolone acetonide in mobile phase.

Study Method

A kinetic study was initiated to test for the release from the pellets. Unstained pellets were used for the drug kinetic assay. For this test, three pellets were wrapped with the coating indicated in results. This wrapping was comprised of shells made using 5% w/v PDLA-A-85K (AP114) glued to each other using a 60% w/v solution of PDLA-A-85K (AP114) in dichloromethane. Three, unwrapped pellets were used as a control series.

Additionally, GMP equivalent to the PDLA-A-85K (AP114) polymer, Resomer R207S from Evonik, has been used to wrap three unstained pellets and a kinetic study was initiated then added to the HPLC data below. These pellets were made in the same procedure as mention above as well as treated in the same manner for testing.

All samples were incubated in 50m1 centrifuge tubes with 10m1 of phosphate buffered saline with 0.05% Tween-20 surfactant (PBST) with 0.1% sodium azide at 37° C. with 100RPM orbital agitation. At each predetermined time interval, a sample was taken from the tube for HPLC analysis and the rest of the solution was discarded and replaced with fresh PBST with 0.1% sodium azide.

Results

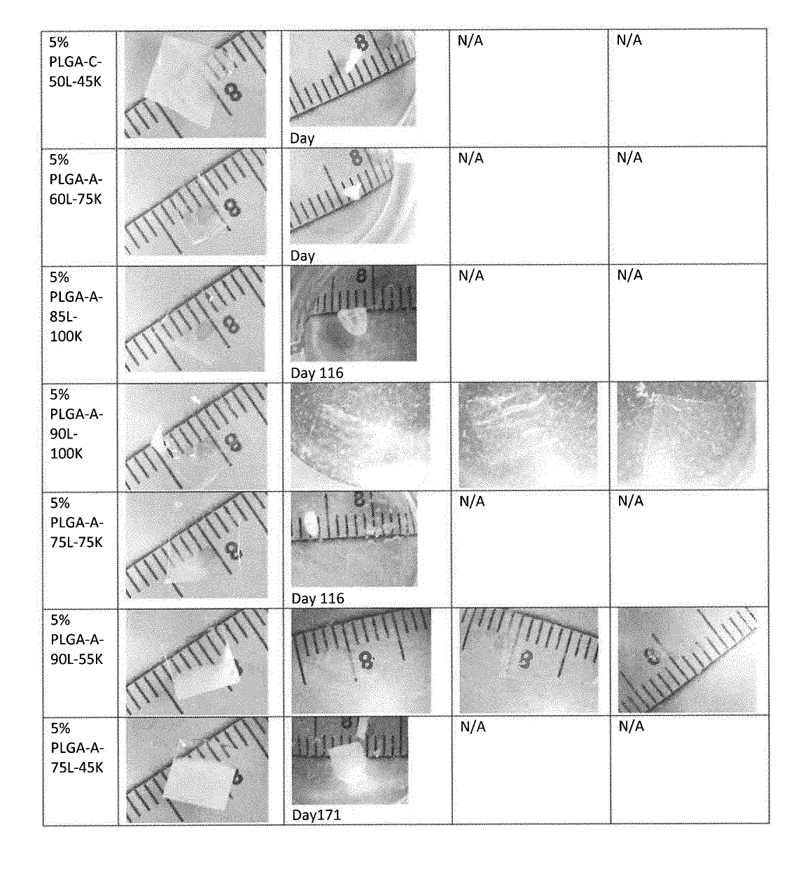

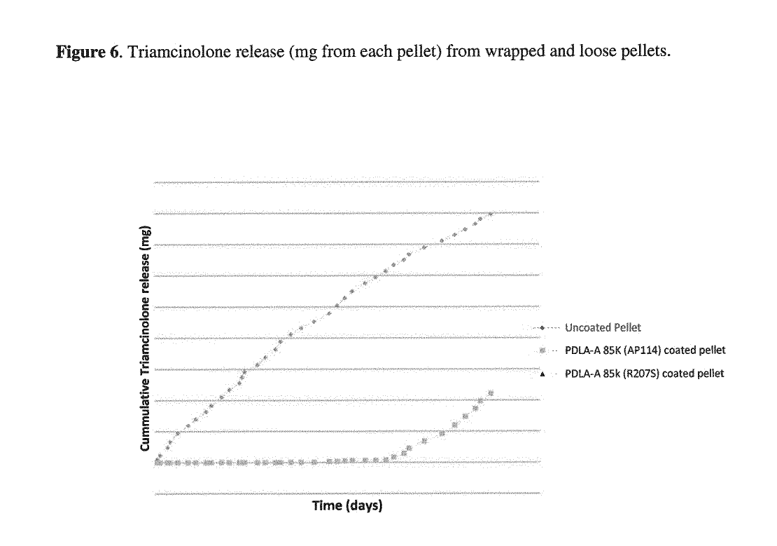

The results of the kinetic study so far are shown below in (FIG. 6). Triamcinolone has been detected in the supernatant of one of the wrapped pellets starting from day 53.

Future Steps

Triplicate repeat tests have been performed with dye-stained pellets for 4-month and 6-month formulations. Additionally, commercially available GMP equivalents to the PDLA-A-85K product. For this, dye release testing has been initiated using Purasorb PDL (cat#1824014) from Corbion, Durect (Lactel) Cat# 136005-2 and Resorner R207S. Prototypes for drug-release testing from a dip-coated Resomer R207S pellet are being generated for subsequent assay.

Further contemplated herein are additional methods of preparing coated and/or encapsulated API-comprising pellets, such as dipping in bio-degradable polymeric not-yet hardened or cured compositions, spraying on a bio-degradable polymeric composition, and the like to prepare time releasable API compositions of choice.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A controlled release and/or sustained release depot drug delivery system, comprising:
(a) a biodegradable polymer capsule and a pha lly efficacious quantity of nattrexone situated within with the biodegradable polymer capsule,
(b) wherein a the quantity of naltrexone coated with biodegradable polymer is effective to be released from the biodegradable polymer capsule over a prolonged period of time in a continuous or rnonophosic manner.

2. (canceled)

3. The controlled release system of claim 1, wherein the prolonged period of time during which naltrexone is released from the polymer extends over a period of time of from about 10 days to about 6 years.

4. (Originally Presented) The controlled release system of claim 1, wherein the biodegradable polymer coating comprises a mixture of polylactic acid and polyglycolic acid and/or copolymers thereof.

5. The controlled release system of claim 4, comprising other effective anti-addiction antagonists.

6. The controlled release system of claim 5, wherein the API is naltrexone.

7. The controlled release system of claim 5, wherein the API is naloxone,

8. The controlled release system of claim 1, wherein the API is disulfiram.

9. (Originally Presented) The controlled release system of claim 1, wherein the biodegradable polymer is selected from the group consisting of poly(lactides), poly(glycolides), poly(lactide-co-glycol ides), poly(lactic acid)s, poly(glycolic acid)s, poly(lactic acid-co-glycolic acid)s, polycaprol actone, polycarbonates, polyesteramides, polyanhydrides, poly(amino acids), polyorthoesters, polycyanoarcylates, poly(p-dioxanone), poly(alkylene oxalates), biodegradable polyurethanes, blends and copolymers thereof.

10. The controlled release system of claim 1, wherein the quantity of naltrexone present is between about 0.01% by wt to about 99.99% by wt.

11. The controlled release system of claim 1 wherein the naltrexone and which is released in an amount at or in the vicinity of the implanted system to act as an effective opioid antagonist is for from about 10 days to about two years.

12. A controlled release system comprising:
(a) a biodegradable polymeric capsule, and;
(b) between about 0.01% by wt and about 99.99% by wt of naltrexone encapsulated within the polymeric capsule, wherein fractional amounts of the API can be released from the polymeric matrix during and after degradation of the polymer capsule over a period of time extending from about 10 days to about two years.

13. A method for making a controlled release system, the method comprising the steps of encapsulating a pharmaceutically effective composition comprising an API in a biodegradable polymer capsule

14. A method for using a continuous release system, the method comprising injection or implantation of a controlled release system which includes an API coated with a biodegradable polymeric matrix, thereby treating a disorder influenced by the APL

15. The system of claim 1, wherein the naltrexone composition further comprises a steroid compound in a steroid effective amount
